SEQUENCE LISTING (13 sequences)

SEQ ID NO: 1 - DNA, 49 bp, Artificial Sequence
  Description of Artificial Sequence Synthetic Nucleic Acid Ligand
tagccaaggtaaccagtacaaggtgctaaacgtaatggcttcggcttac

SEQ ID NO: 2 - DNA, 32 bp, Artificial Sequence
  Description of Artificial Sequence Synthetic Nucleic Acid Ligand
gtagtcactggttggtgaggttgggtgactac

SEQ ID NO: 3 - DNA, 37 bp, Artificial Sequence
  Description of Artificial Sequence Synthetic Nucleic Acid Ligand
gcttagtccgtggtagggcaggttggggtgactaagc

SEQ ID NO: 4 - DNA, 32 bp, Artificial Sequence; identical to SEQ ID NO: 2
  Description of Artificial Sequence Synthetic Nucleic Acid Ligand

SEQ ID NO: 5 - DNA, 38 bp, Artificial Sequence
  Description of Artificial Sequence Synthetic Nucleic Acid Ligand
gtagtcactggttggtgaggttgggtgactactttttt

SEQ ID NO: 6 - DNA, 32 bp, Artificial Sequence; identical to SEQ ID NO: 2
  Description of Artificial Sequence Synthetic Nucleic Acid Ligand

SEQ ID NO: 7 - DNA, 38 bp, Artificial Sequence
  Description of Artificial Sequence Synthetic Nucleic Acid Ligand
ttttttgtagtcactggttggtgaggttgggtgactac

SEQ ID NO: 8 - DNA, 70 bp, Artificial Sequence
  Description of Artificial Sequence Synthetic Nucleic Acid Ligand
gtagtcactggttggtgaggttgggtgactacttttttcatcagtgggttggagtggttggtcactgatg

SEQ ID NO: 9 - DNA, 37 bp, Artificial Sequence; identical to SEQ ID NO: 3
  Description of Artificial Sequence Synthetic Nucleic Acid Ligand

SEQ ID NO: 10 - DNA, 43 bp, Artificial Sequence
  Description of Artificial Sequence Synthetic Nucleic Acid Ligand
gcttagtccgtggtagggcaggttggggtgactaagctttttt

SEQ ID NO: 11 - DNA, 37 bp, Artificial Sequence; identical to SEQ ID NO: 3
  Description of Artificial Sequence Synthetic Nucleic Acid Ligand

SEQ ID NO: 12 - DNA, 42 bp, Artificial Sequence
  Description of Artificial Sequence Synthetic Nucleic Acid Ligand
tttttgcttagtccgtggtagggcaggttggggtgactaagc

SEQ ID NO: 13 - DNA, 74 bp, Artificial Sequence
  Description of Artificial Sequence Synthetic Nucleic Acid Ligand
gcttagtccgtggtagggcaggttggggtgactaagccgaatcagtggggttggacgggatggtgcctgattcg

(19) United States
(12) Patent Application Publication
(10) Pub. No.: US 2002/0037506 A1
(43) Pub. Date: Mar. 28, 2002

(54) Aptamer based two-site binding assay

(76) Inventors: Yun Lin, Louisville, CO (US); James R. Heil, Boulder, CO (US); Sumedha D. Jayasena, Boulder, CO (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 09/681,508

(22) Filed: Apr. 18, 2001

Related U.S. Application Data
(60) Provisional application No. 60/198,016, filed on Apr. 18, 2000.

(51) Int. Cl. C12Q 1/68

(52) U.S. Cl. 435/6

(57) ABSTRACT

This invention discloses a novel aptamer based two-site binding sandwich assay, employing nucleic acid ligands as capture and/or reporter molecules.

### CROSS REFERENCE TO RELATED APPLICATIONS

This application claims priority from U.S. Provisional Patent Application Serial No. 60/198,016, filed Apr. 18, 2000, entitled “Two-Site Binding Assay Exclusively Based on Aptamers: Multiplexed Analysis of Proteins in Flow Cytometry.”

### FIELD OF THE INVENTION

Described herein are methods for performing a novel aptamer based sandwich assay employing nucleic acid ligands as capture and/or reporter molecules. The method utilized herein for identifying and preparing said nucleic acid ligands is called SELEX, an acronym for Systematic Evolution of Ligands by EXponential enrichment. The invention includes high-affinity nucleic acid ligands which bind to various targets that can act as capture molecules and/or reporter molecules in a sandwich type format for the detection of targets in biological fluids, cell culture media and industrial processes and further determination of the target quantity found in the substance. Specifically disclosed are assays wherein nucleic acid ligands to human α-thrombin are used to capture and/or to detect the captured target compound. Also specifically disclosed is an assay based on a nucleic acid ligand and Protein A to detect human L-Selectin-Ig chimera. The nucleic acid ligand based sandwich assays, designed on two different types of beads that can be readily analyzed in flow cytometry, allow multiplexed analysis of a mixture of target proteins in a single tube.

### BACKGROUND OF THE INVENTION

The Systematic Evolution of Ligands by Exponential Enrichment (SELEX) process is a method for the in vitro evolution of nucleic acid molecules with highly specific binding to target molecules and is described in U.S. patent application Ser. No. 07/536,428, filed Jun. 11, 1990, entitled “Systematic Evolution of Ligands by EXponential Enrichment,” now abandoned, U.S. Pat. No. 5,475,096, entitled “Nucleic Acid Ligands,” and U.S. Pat. No. 5,270,163 (see also WO 91/19813), entitled “Methods for Identifying Nucleic Acid Ligands,” each of which is specifically incorporated herein by reference in its entirety. Each of these applications, collectively referred to herein as the SELEX Patent Applications, describes a fundamentally novel method for making a nucleic acid ligand to any desired target molecule.

The SELEX process provides a class of products which are referred to as nucleic acid ligands or aptamers, each having a unique sequence, and which has the property of binding specifically to a desired target compound or molecule. Each SELEX-identified nucleic acid ligand is a specific ligand of a given target compound or molecule. The SELEX process is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets. The SELEX method applied to the application of high affinity binding involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand enriched mixture of nucleic acids, then reiterating the steps of binding, partitioning, dissociating and amplifying through as many cycles as desired to yield highly specific high affinity nucleic acid ligands to the target molecule.

It has been recognized by the present inventors that the SELEX method demonstrates that nucleic acids as chemical compounds can form a wide array of shapes, sizes and configurations, and are capable of a far broader repertoire of binding and other functions than those displayed by nucleic acids in biological systems.

The basic SELEX method has been modified to achieve a number of specific objectives. For example, U.S. patent application Ser. No. 07/960,093, filed Oct. 14, 1992, now abandoned, and U.S. Pat. No. 5,707,796, both entitled “Method for Selecting Nucleic Acids on the Basis of Structure,” describe the use of the SELEX process in conjunction with gel electrophoresis to select nucleic acid molecules with specific structural characteristics, such as bent DNA. U.S. patent application Ser. No. 08/123,935, filed Sep. 17, 1993, entitled “Photoselection of Nucleic Acid Ligands,” now abandoned, U.S. Pat. No. 5,763,177 and U.S. Pat. No. 6,011,577, both entitled “Systematic Evolution of Ligands by Exponential Enrichment: Photoselection of Nucleic Acid Ligands and Solution SELEX,” describe a SELEX based method for selecting nucleic acid ligands containing photoreactive groups capable of binding and/or photocrosslinking to and/or photoinactivating a target molecule. U.S. Pat. No. 5,580,737, entitled “High-Affinity Nucleic Acid Ligands That Discriminate Between Theophylline and Caffeine,” describes a method for identifying highly specific nucleic acid ligands able to discriminate between closely related molecules, which can be non-peptidic, termed Counter-SELEX. U.S. Pat. No. 5,567,588, entitled “Systematic Evolution of Ligands by EXponential Enrichment: Solution SELEX,” describes a SELEX-based method which achieves highly efficient partitioning between oligonucleotides having high and low affinity for a target molecule. In U.S. Pat. No. 5,496,938, methods are described for obtaining improved nucleic acid ligands after the SELEX process has been performed. This patent, entitled “Nucleic Acid Ligands to HIV-RT and HIV-1 Rev,” is specifically incorporated herein by reference. U.S. Pat. No. 5,705,337, entitled “Systematic Evolution of Ligands by Exponential Enrichment: Chemi-SELEX,” describes methods for covalently linking a ligand to its target.

One potential problem encountered in the diagnostic use of nucleic acids is that oligonucleotides in their phosphodiester form may be quickly degraded in body fluids by intracellular and extracellular enzymes, such as endonucleases and exonucleases, before the desired effect is manifest. Certain chemical modifications of the nucleic acid ligand can be made to increase the in vivo stability of the nucleic acid ligand or to enhance or to mediate the delivery of the nucleic acid ligand. See, e.g., U.S. patent application Ser. No. 08/117,991, filed Sep. 8, 1993, now abandoned and U.S. Pat. No. 5,660,985, both entitled “High Affinity Nucleic Acid Ligands Containing Modified Nucleotides,” and U.S. patent application Ser. No. 09/362,578, filed Jul. 28, 1999, entitled “Transcription-free SELEX,” each of which is specifically incorporated herein by reference in its entirety. Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases, isocytidine and isoguanidine and the like. Modifications can also include 3′ and 5′ modifications such as capping. In preferred embodiments of the instant invention, the nucleic acid ligands are RNA molecules that are 2′-fluoro (2′-F) modified on the sugar moiety of pyrimidine residues.

The SELEX method encompasses combining selected oligonucleotides with other selected oligonucleotides and non-oligonucleotide functional units as described in U.S. Pat. No. 5,637,459, entitled “Systematic Evolution of Ligands by EXponential Enrichment: Chimeric SELEX,” and U.S. Pat. No. 5,683,867, entitled “Systematic Evolution of Ligands by EXponential Enrichment: Blended SELEX,” respectively. These applications allow the combination of the broad array of shapes and other properties, and the efficient amplification and replication properties, of oligonucleotides with the desirable properties of other molecules.

The SELEX method further encompasses combining selected nucleic acid ligands with lipophilic compounds or non-immunogenic, high molecular weight compounds in a diagnostic or therapeutic complex as described in U.S. Pat. No. 6,011,020, entitled “Nucleic Acid Ligand Complexes.” Each of the above described patent applications which describe modifications of the basic SELEX procedure are specifically incorporated by reference herein in their entirety.

The modifications can be pre- or post-SELEX process modifications. Pre-SELEX process modifications yield nucleic acid ligands with both specificity for their SELEX target and improved in vivo stability. Post-SELEX process modifications made to 2′-OH nucleic acid ligands can result in improved in vivo stability without adversely affecting the binding capacity of the nucleic acid ligand. Other modifications are known to one of ordinary skill in the art. Such modifications may be made post-SELEX process (modification of previously identified unmodified ligands) or by incorporation into the SELEX process.

Technologies that allow molecular detection and quantification have become an important aspect of research and clinical diagnostics. As a result, these technologies are constantly being evolved to improve their performance, as well as to cater to new analytes. Molecules that recognize others with extreme specificity and high-affinity are important for a wide range of applications, including molecular diagnostics. Antibodies fulfill this role in immunoassays. Nucleic acids probe-based diagnostic assays rely on the Watson-Crick base pairing that dictates specific and tight binding of complementary oligonucleotide strands. Recent advancement of research has led to the discovery of a class of oligonucleotides referred to as aptamers that can recognize molecules other than nucleic acids with high-affinity and specificity. (Tuerk and Gold (1990) Science 249: 505-510; Ellington and Szostak (1990) Nature 346:818-822; Gold et al. (1995) Annu. Rev. Biochem. 64:763-797; Gold (1995) J. Biol. Chem. 270:13581-13584). Consequently, aptamers have the potential to fulfill the role that antibodies play in diagnostics applications. Aptamers are being identified from random sequence oligonucleotide libraries that are subjected to iterative cycles of in vitro selection and amplification. The selection is carried out on the basis of affinity toward a target molecule of interest under the conditions set forth by the user. Hence, aptamers represent a class of oligonucleotide molecules that could bind target molecules of interest with high-affinity and specificity under a variety of environmental conditions that are not restricted to in vivo conditions, a feature that is generally different from that of antibodies. Hence certain applications that have not been possible with antibodies may become feasible with aptamers.

Several features of aptamers make them especially attractive for diagnostic applications. These include, but are not limited to, the following. 1) The identification process of aptamers does not depend on animals or in vivo conditions, expanding their applications to molecules that are not well tolerated by animals in generating antibodies. 2) In vitro conditions can be manipulated to change the properties of aptamers on demand. For example, aptamers can be identified to bind their targets under specific salt ions, temperature and pH conditions. 3) Aptamers are produced by chemical synthesis, an accurate and reproducible process that generates materials with little or no batch to batch variation. 4) Reporter molecules, as well as functional groups that could subsequently be activated to conjugate aptamers to molecules of choice can be incorporated during chemical synthesis with high efficiency. 5) Denaturation of aptamers is reversible and upon denaturation, functional aptamers can be regenerated within minutes. 6) Aptamers are stable to long-term storage and can be transported at ambient temperature.

Recently, the performance of aptamers have been tested in a variety of diagnostic applications, and the results of these experiments have strengthened the promise of aptamers to become key in future diagnostics (reviewed in Jayasena (1999) Clin. Chem. 45: 1628-1650). However, as with any other emerging technology, aptamers should also undergo various tests and comparisons with existing technologies before they become embraced as the reagent of choice. Hence, testing of aptamers in every possible application in combination with or in lieu of antibodies should be encouraged. Results of such investigations are expected to guide the future of aptamers. Although aptamers have been tested in different diagnostic platforms, a sandwich assay that is completely based on aptamers has not yet been described. Two-site binding assays are considered to have enhanced specificity provided by the second ligand binding to the same target. Two-site binding assays employing aptamers in combination with antibodies have been described (Davis et al. (1998) Nucleic Acids Res. 26:3915-3924; Drolet et al. (1996) Nature Biotechnol. 14:1021-1025). The present invention, however, describes a two-site binding assay exclusively based on aptamers and multiplexed analysis of targets using aptamer-based assays. The present invention also describes the detection of a specific target within a complex background using the sandwich assay configured on a membrane, an approach that is important for proteomics applications. Overall, the results of these experiments in combination with the existing examples of applications of aptamers in various diagnostics formats further strengthen the future of aptamers as a useful class of reagents for diagnostics.

### SUMMARY OF THE INVENTION

The present invention includes methods for performing novel immunoassays employing nucleic acid ligands. More specifically, the present invention includes a novel sandwich-type assay based on two aptamers that recognize two independent sites on a target molecule. In particular the present invention provides a method for detecting the presence of a target compound in a substance which may contain said target compound comprising: a) exposing a substance which may contain said target compound to a capture molecule capable of binding to said target molecule, wherein said capture molecule is immobilized on a solid support; b) removing the remainder of said substance from said capture molecule:target molecule complex; c) adding to said capture molecule:target molecule complex a reporter molecule capable of binding to said target molecule; and d) detecting said capture molecule:target molecule:reporter molecule complex; wherein said capture molecule and reporter molecule both are a nucleic acid ligand to said target molecule. In a preferred embodiment, the method utilized for identifying and preparing said nucleic acid ligands is called SELEX, an acronym for Systematic Evolution of Ligands by EXponential enrichment. In a preferred embodiment the solid support is selected from microsphere particles or a membrane.

In one embodiment, the present invention describes a novel sandwich assay based on two aptamers that recognize human α-thrombin at two independent binding sites. One of the aptamers functions as a capture probe and is attached to microsphere particles and the second aptamer is conjugated to fluorescein and serves as a reporter probe in the two-site binding assay. The two-site binding assay configured on microsphere particles is carried out in one step and analyzed by flow cytometry. The assay specifically detects human α-thrombin in buffer as well as in biological fluids.

In a second embodiment, using an aptamer that recognizes human L-Selectin and Protein A, an assay that specifically detects human L-Selectin-Ig fusion protein is described. The aptamer is immobilized on microsphere particles and captures L-Selectin-Ig chimera, whereas Protein A-Alexa conjugate that binds to the immunoglobulin domain detects L-Selectin-Ig bound to particles. Similar to the antibody-based one-step sandwich assays, the aptamer-based assays also exhibit a “hook effect” at high target concentrations.

The two sandwich assays configured on two distinct microsphere particles that can be analyzed separately, but simultaneously in flow cytometry, allow multiplexed analysis of cognate targets in a single tube. The sandwich assay aimed to detect thrombin is replicated on a membrane to specifically capture and detect thrombin in a complex background such as biological fluids. The latter is a demonstration towards an aptamer-based chip for analyzing protein expression in an organism, a prelude to proteomics.

### BRIEF DESCRIPTION OF DRAWINGS

FIG. 1A illustrates the primary and secondary structures of the two aptamers (SEQ ID NOS:2 and 3) used to design the sandwich assay for detecting human α-thrombin. The two aptamers have identical folded structure; a G-quartet with a duplexed end. MC-Aptamer (SEQ ID NO:2) is based on sequence 9 described by Macaya et al. ((1995) Biochemistry 34:4478-4492) with minor modifications; the stem is truncated from 8 base pairs to 6 base pairs and non base-paired nucleotides were increased 1 to 2 in one side and zero to three in the other. Such changes seem to be tolerated by the aptamers belonging to this class. In this study, the length of the duplexed region of the DT-Aptamer (SEQ ID NO:3) described by Tasset et al ((1997) J. Mol. Biol. 272:688-698) was increased from 4 base pairs to 8 base pairs to provide more stability in the duplex.

FIG. 1B illustrates a schematic representation of the sandwich assay. The capture aptamer synthesized with biotin at the 3′ terminus is immobilized on streptavidin present on the surface of Lumavidin™ microsphere particles or beads. These beads are simultaneously incubated with thrombin and the detector aptamer labeled with fluorescein to form the sandwich on the bead. Beads that become fluorescent upon the formation of the sandwich are detected in a flow cytometer.

FIG. 2 illustrates the detection of thrombin by the aptamer-based sandwich assay. These assays were carried out using MC-5′-LNK-F (SEQ ID NO:12) as the reporter aptamer. The fluorescence signal generated on DT-beads is shown upon incubating with either thrombin (filled square) or L-Selectin (open circle) and the reporter aptamer. Closed circles (filled circle) indicate the signal generated in the assay when DT-beads were replaced with LS-beads that contain a DNA aptamer specific for L-Selectin. Data points (filled square) up to the end of the plateau were included for curve fitting.

FIG. 3 depicts graphically an analysis of different reporter aptamer constructs in two configurations of the aptamer-based sandwich assay. Primary structures of these reporter constructs are shown in Table 1. FIG. 3A illustrates an analysis of the fluorescence signal generated as a function of the concentration of different reporter constructs based on MC-aptamer. Varying concentrations of each reporter aptamer were incubated with a fixed number of DT-beads and 30 nM thrombin. FIG. 3B illustrates an analysis of the fluorescence signal generated as a function of the concentration of different reporter constructs based on the DT-aptamer. Varying concentrations of each reporter aptamer were incubated with a fixed number of MC-beads and 30 nM thrombin.

FIG. 4 illustrates graphically the detection of thrombin in human urine (filled circle) and in human plasma (open circle) using an aptamer-based sandwich assay. DT-beads and MC-5′-LNK-F aptamer were mixed with varying concentrations of thrombin spiked into either human plasma or human urine. Mixing of these components diluted the biological fluid by 50%.

FIG. 5A depicts an analysis of the fluorescence signal generated on LS-beads upon incubating with 10 nM L-Selectin-Ig chimera and increasing concentrations of Protein A-Alexa conjugate.

FIG. 5B depicts the detection of L-Selectin-Ig chimera using the sandwich assay that utilizes an aptamer immobilized on beads (LS beads) to capture L-Selectin-Ig chimera and Protein A-Alexa to detect the captured chimeric protein through the Ig tail. Closed circles (filled circle) indicate the fluorescent signal generated with varying concentrations of L-Selectin-Ig chimera in the assay, whereas the open circles (open circle) show the signal generated with CTLA-4-Ig chimera, another protein construct with an Ig tail.

FIGS. 6A-C illustrate a multiplexed analysis of thrombin and L-Selectin using sandwich assays that employ aptamers. The two assays were designed separately to detect thrombin and L-Selectin and carried out in a single tube. Tubes contained a mixture of two types of beads, DT-beads to capture thrombin and LS-beads to capture L-Selectin-Ig chimera, and a mixture of reporter probes, MC-5′-LNK-F to detect thrombin and Protein A-Alexa to detect the Ig tail in L-Selectin-Ig chimera. FIG. 6A shows fluorescence signals observed on two types of beads when increasing concentration of L-Selectin-Ig chimera alone is present. Open circles (open circle) indicate the fluorescence measured on LS beads, whereas closed circles (filled circle) show fluorescence on DT-beads. FIG. 6B indicates fluorescent signals observed on two types of beads when increasing concentration of thrombin alone is present. Open circles (open circle) indicate the fluorescence measured on LS beads, whereas closed circles (filled circle) show fluorescence on DT-beads. FIG. 6C shows fluorescent signals observed on two types of beads when increasing concentrations of thrombin and L-Selectin-Ig are present. Open circles (open circle) indicate the fluorescence measured on LS beads, whereas closed circles (filled circle) show fluorescence on DT-beads. The scale on the left abscissa corresponds to the signal indicated in closed circles, whereas the scale on the right abscissa corresponds to the signal shown in open circles.

FIG. 7A is a schematic illustration of the two-step sandwich assay configured on a nylon membrane. DT-aptamer that serves as the capture is attached to carboxylic groups on the membrane through a primary amine group at its 3′ end. Thrombin spiked either into the assay buffer or into human urine (or plasma) is added and the membrane is washed to remove thrombin that was not captured by the aptamer. Detector probe is then added to form a sandwich on the membrane. Excess reporter probe is washed away before detection of the signal.

FIG. 7B depicts graphically the detection of thrombin by the two-step sandwich assay on a nylon membrane. Detection of thrombin spiked into 1) the assay buffer (open circle), 2) the assay buffer containing a mixture of proteins; 10 nM human neutrophil elastase, 10 nM human thyroid stimulating hormone and 0.2% (w/v) human serum albumin (filled circle), 3) human urine (filled square) and 4) human plasma (filled triangle). Open squares (open square) indicate the signal generated on the plain membrane without immobilized capture aptamer.

### DETAILED DESCRIPTION OF THE INVENTION

The present invention describes methods for performing novel immunoassays employing nucleic acid ligands. More specifically, the present invention describes a novel sandwich-type assay based on two aptamers that recognize two independent sites on a target molecule. In the preferred embodiments, the nucleic acid ligand is a single stranded nucleic acid ligand identified using the SELEX methodology.

Various terms are used herein to refer to aspects of the present invention. To aid in the clarification of the description of the components of this invention, the following definitions are provided.

As used herein a “nucleic acid ligand” is a non-naturally occurring nucleic acid having a desirable action on a target. Nucleic acid ligands are often referred to as “aptamers.” A desirable action includes, but is not limited to, binding of the target, catalytically changing the target, reacting with the target in a way which modifies/alters the target or the functional activity of the target, covalently attaching to the target as in a suicide inhibitor, facilitating the reaction between the target and another molecule. In a preferred embodiment, the action is specific binding affinity for a target molecule, such target molecule being a three dimensional chemical structure other than a polynucleotide that binds to the nucleic acid ligand through a mechanism which predominantly depends on Watson/Crick base pairing or triple helix binding, wherein the nucleic acid ligand does not have the known physiological function of being bound by the target molecule.

As used herein a “candidate mixture” is a mixture of nucleic acids of differing sequence from which to select a desired ligand. The source of a candidate mixture can be from naturally-occurring nucleic acids or fragments thereof, chemically synthesized nucleic acids, enzymatically synthesized nucleic acids or nucleic acids made by a combination of the foregoing techniques. In a preferred embodiment, each nucleic acid has fixed sequences surrounding a randomized region to facilitate the amplification process.

As used herein, “nucleic acid” means either DNA, RNA, single-stranded or double-stranded, and any chemical modifications thereof. Modifications include, but are not limited to, those which provide other chemical groups that incorporate additional charge, polarizability, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

“SELEX” methodology involves the combination of selection of nucleic acid ligands that interact with a target in a desirable manner, for example binding to a protein, with amplification of those selected nucleic acids. Optional iterative cycling of the selection/amplification steps allows selection of one or a small number of nucleic acids which interact most strongly with the target from a pool which contains a very large number of nucleic acids. Cycling of the selection/amplification procedure is continued until a selected goal is achieved. The SELEX methodology is described in the SELEX Patent Applications.

“SELEX target” or “target” means any compound or molecule of interest for which a ligand is desired. A target can be a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, etc. without limitation.

As used herein, “solid support” is defined as any surface to which molecules may be attached through either covalent or non-covalent bonds. This includes, but is not limited to, membranes, microsphere particles, such as Lumavidin™ or LS-beads, microtiter plates, magnetic beads, charged paper, nylon, Langmuir-Bodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is also contemplated. This includes surfaces with any topology, including, but not limited to, spherical surfaces and grooved surfaces.

Note, that throughout this application various citations are provided. Each citation is specifically incorporated herein in its entirety by reference.

In the preferred embodiment, the nucleic acid ligands/aptamers of the present invention are derived from the SELEX methodology. The SELEX process is described in U.S. patent application Ser. No. 07/536,428, entitled “Systematic Evolution of Ligands by Exponential Enrichment,” now abandoned, U.S. Pat. No. 5,475,096, entitled “Nucleic Acid Ligands,” and U.S. Pat. No. 5,270,163 (see also WO 91/19813), entitled “Methods for Identifying Nucleic Acid Ligands.” These applications, each specifically incorporated herein by reference, are collectively called the SELEX Patent Applications.

The SELEX process provides a class of products that are nucleic acid molecules, each having a unique sequence, and each of which has the property of binding specifically to a desired target compound or molecule. Target molecules are preferably proteins, but can also include among others carbohydrates, peptidoglycans and a variety of small molecules. SELEX methodology can also be used to target biological structures, such as cell surfaces or viruses, through specific interaction with a molecule that is an integral part of that biological structure.

In its most basic form, the SELEX process may be defined by the following series of steps.

1. A candidate mixture of nucleic acids of differing sequence is prepared. The candidate mixture generally includes regions of fixed sequences (i.e., each of the members of the candidate mixture contains the same sequences in the same location) and regions of randomized sequences. The fixed sequence regions are selected either: (a) to assist in the amplification steps described below; (b) to mimic a sequence known to bind to the target; or (c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100 percent).

2. The candidate mixture is contacted with the selected target under conditions favorable for binding between the target and members of the candidate mixture. Under these circumstances, the interaction between the target and the nucleic acids of the candidate mixture can be considered as forming nucleic acid-target pairs between the target and those nucleic acids having the strongest affinity for the target.

3. The nucleic acids with the highest affinity for the target are partitioned from those nucleic acids with lesser affinity to the target. Because only an extremely small number of sequences (and possibly only one molecule of nucleic acid) corresponding to the highest affinity nucleic acids exist in the candidate mixture, it is generally desirable to set the partitioning criteria so that a significant amount of the nucleic acids in the candidate mixture (approximately 5-50%) are retained during partitioning.

4. Those nucleic acids selected during partitioning as having the relatively higher affinity for the target are then amplified to create a new candidate mixture that is enriched in nucleic acids having a relatively higher affinity for the target.

5. By repeating the partitioning and amplifying steps above, the newly formed candidate mixture contains fewer and fewer unique sequences, and the average degree of affinity of the nucleic acids to the target will generally increase. Taken to its extreme, the SELEX process will yield a candidate mixture containing one or a small number of unique nucleic acids representing those nucleic acids from the original candidate mixture having the highest affinity to the target molecule.

The SELEX Patent Applications describe and elaborate on this process in great detail. Included are targets that can be used in the process; methods for partitioning nucleic acids within a candidate mixture; and methods for amplifying partitioned nucleic acids to generate an enriched candidate mixture. The SELEX Patent Applications also describe ligands obtained to a number of target species, including both protein targets where the protein is and is not a nucleic acid binding protein.

SELEX provides high affinity ligands of a target molecule. This represents a singular achievement that is unprecedented in the field of nucleic acids research. Affinities of SELEX-derived nucleic acid ligands often lie in the same range observed with structurally large monoclonal antibodies.

In one embodiment, it is preferred that the nucleic acid ligand: 1) binds to the target in a manner capable of achieving the desired effect on the target; 2) be as small as possible to obtain the desired effect; 3) be as stable as possible; and 4) be a specific ligand to the chosen target. In most situations, it is preferred that the nucleic acid ligand have the highest possible affinity to the target.

Aptamers are emerging as a class of molecules that could fulfill the need for molecular recognition in a variety of applications. They have been tested in different application formats with remarkable success. In these experiments aptamers have been used either in lieu of, or in combination with antibodies. Compared to one-site binding assays such as fluorescence polarization assays, the two-site binding assay, or commonly referred to as a sandwich assay, has the advantage of enhanced specificity provided by the second dimension of ligand binding. Concerted binding of two ligands to the same molecular target at non-competing sites is required to develop a two-site binding assay. Typically, SELEX experiments have led to the identification of high-affinity aptamers to their targets, and these aptamers could fall into different families based on their primary and secondary structures. However, in almost all cases, aptamers identified to recognize a given target tend to recognize either an identical site or overlapping sites, regardless of their folded structures. This could be due to the way in which a typical SELEX experiment is carried out to identify aptamers with the highest affinity for a given target. In other words, SELEX experiments are pushed to the limit of affinity saturation of enriched libraries, a condition that may direct aptamers to the site of highest affinity on a given target.

There are, however, a few known exceptions to the above observation. Aptamers that bind two different regions of a target were isolated in a selection experiment that employed the gag polyprotein of HIV-1. (Lochrie et al. (1997) Nucleic Acid Res. 25:2902-2910). In this experiment two classes of aptamers were identified; one class recognized the nucleocapsid protein, whereas the other bound to the matrix protein within the gag polyprotein. A second example in which aptamers that bind two different regions of a target were isolated are the two aptamers to thrombin used in the present study. Presentation of the same target, thrombin, to DNA libraries by two independent research groups in two different ways resulted in the isolation of two different aptamers. (Macaya et al. (1995) Biochemistry 34:4478-4492; Tasset et al. (1997) J. Mol. Biol. 272:688-698). Tasset et al. exposed thrombin in solution to the DNA library, whereas Macaya et al. used thrombin presented on concanavalin A agarose support during the SELEX process. The binding of thrombin to concanavalin A must have hindered the otherwise preferred binding site on the protein, resulting in the isolation of an aptamer which binds to a second region on the protein. This illustrates one approach for identifying two different aptamers binding to the same target at different or non-overlapping binding sites. Two other possible approaches for identifying ligands binding to non-overlapping sites include. 1. Searching for aptamers in an intermediate cycle of a SELEX process. This may lead to the identification of aptamers with lower affinity that bind to different sites on a target molecule. 2. Carrying out a SELEX experiment using a target presented on an already selected aptamer. This approach could potentially direct emerging aptamers to a site different from the one already occupied by the primary aptamer. Alternatively, the emerging secondary aptamers may recognize the primary aptamer-target complex. The latter would be an attractive approach for identifying two aptamers for small molecule targets.

Interactions of small molecule targets with their cognate aptamers have been studied by nuclear magnetic resonance spectroscopy. (Feigon et al. (1996) Chem. & Biol. 3:611-617 28; Hsiung and Patel (1996) Nature Struct. Biol. 3:1046-1050; Patel et al. (1997) J. Mol. Biol. 272:645-664; Yang et al. (1996) Science 272:1343-1347; Zimmermann et al. (1997) Nature Struct. Biol. 4:644-649). These studies revealed that targets are buried in binding pockets formed by cognate aptamers. Within a binding pocket, a target molecule makes numerous contacts with its cognate aptamer through electrostatic, hydrogen bonding and hydrophobic interactions. (See also, Hermann and Patel (2000) Science 287:820-825). Aptamers engulf their targets in such a way that little or no surface of targets is available to interact with a secondary ligand. Hence, secondary aptamers may be generated to bind to the primary aptamer-target complexes to design sandwich assays for small molecules with enhanced specificity.

Often, immunoassays are in a sandwich-type format. Unless used in a homogeneous detection assay, such as fluorescence anisotropy, most widely used sandwich assays are based on the binding of two molecules, a reporter molecule and a capture molecule, to an analyte or target. In a sandwich assay, typically, the capture molecule is attached to a solid support. A substance that may contain the target compound is applied and allowed to react with the capture molecule. After washing, the reporter molecule is added to react with the target and the detection system indicates that an interaction has occurred. The target or antigen is, thus, “sandwiched” between the two layers of molecules, traditionally antibodies. This technique is adaptable to the procedure described herein.

The present invention provides a method for detecting the presence of a target compound in a substance which may contain said target compound comprising: a) exposing a substance which may contain said target compound to a capture molecule capable of binding to said target molecule, wherein said capture molecule is immobilized on a solid support; b) removing the remainder of said substance from said capture molecule:target molecule complex; c) adding to said capture molecule:target molecule complex a reporter molecule capable of binding to said target molecule; and d) detecting said capture molecule:target molecule:reporter molecule complex; wherein said capture molecule and reporter molecule both are a nucleic acid ligand to said target molecule. In the preferred embodiment of the invention, both the capture molecule and the reporter molecule are nucleic acid ligands.

The capture molecule must bind to the target molecule to form a capture molecule:target molecule complex. The reporter molecule must also bind to the target molecule, but additionally must comprise a detection system wherein a capture molecule:target molecule:reporter molecule complex can be identified. The reporter molecule comprises a detection system that comprises a wide array of known chemical entities. The detection system can be an enzyme, a fluorophore, a radiolabel, etc. The various detection systems are well known to those skilled in the art. In the preferred embodiment, the reporter molecule comprises ligands labeled with fluorescein or Alexa as reporter ligands. Alternatively, enzymes such as alkaline phosphatase that could subsequently be used to generate fluorescence or chemiluminescence can also be conjugated to the reporter ligand. Such enzyme conjugates are expected to further enhance the sensitivity of the assay. Enzymes or other appropriate reporter groups can be introduced to reporter ligands either directly or indirectly, for example, through either biotin-streptavidin interaction (Davis et al. (1996) Nucleic Acids Res. 24:702-706) or antigen-antibody interaction. (Drolet et al. (1996) Nature Biotechnol. 14:1021-1025).

In a preferred embodiment the two-site assay is analyzed by flow cytometry. The use of flow cytometry allows simultaneous multi-parameter analysis on cells or particles. An important feature of flow cytometry is the ability to simultaneously detect single-particle light scatter and fluorescence emission at three different wavelengths. As a result, assays carried out by flow cytometry can be configured in several ways for multiplexed analyses. Particles of different sizes can be distinguished on light scatter mode and their fluorescence can be measured simultaneously. On each particle, binding of three different ligands attached to three distinct fluorophores can be analyzed simultaneously. Alternatively, particles of identical size, but emitting different ratios of fluorescence at two wavelengths can be distinguished simultaneously. Particles dyed with sixty-four different ratios of red and orange fluorescent dyes have been simultaneously identified in flow cytometry using FlowMetrix™ software. (Kettman et al. (1998) Cytometry 33:234-243). This configuration leaves single-color reporter ligand for analysis. Multiplexed analysis using the FlowMetrix™ system has been described for nucleic acid targets (Fulton et al. (1997) Clin. Chem. 43:1749-1756; Smith et al. (1998) Clin. Chem. 44:2054-2056), as well as for other targets (Oliver et al. (1998) Clin. Chem. 44:2057-2060). Six sets of beads dyed with a fluorescent dye to discrete intensities have been used for multiplexed analysis of six cytokines without additional software, but using a FACScan flow cytometer alone. (Chen et al. (1999) Clin. Chem. 45:1693-1694). These studies indicate the feasibility of multiplexed target analysis in flow cytometry. In the present study, the Luminex FlowMetrix™ software program was used to investigate the feasibility of multiplexed analysis of aptamer-based sandwich assays configured on two different types of microspheres dyed with two different ratios of red/orange fluorescent dyes.

The preferred use of the method of the present invention is for the detection of target compounds for the clinical diagnosis of physiologic conditions, the immunoassays will most frequently be contacted with a substance that may contain a target compound. The substance is usually a biological material that may or may not contain the target compound of interest. Such biological materials include blood, plasma, serum, sputum, urine, semen, cerebrospinal fluid, bronchial aspirate, and macerated tissue. The assays of the present invention are useful for both human and veterinary diagnostics. Other samples that may be assayed using the methods described herein include foods and environmental discharges such as liquid wastes.

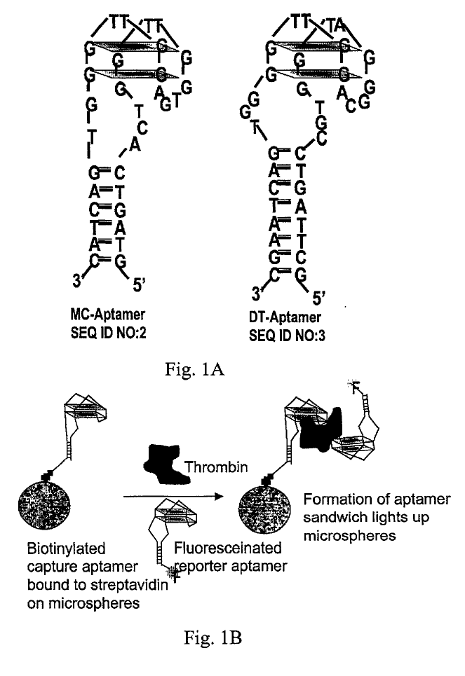

To design a sandwich assay exclusively based on aptamers, two different aptamer sequences were chosen. As noted above, these sequences were isolated and characterized to bind human α-thrombin by two independent research groups. FIG. 1A shows the primary and predicted secondary structures of these two aptamers. MC-aptamer (SEQ ID NO:2) was identified by Macaya et al. ((1995) Biochemistry 34:4478-4492; oligonucleotide 9) in a SELEX experiment using an affinity matrix constructed by immobilizing thrombin on a concanavalin A-agarose support. Using nuclear magnetic resonance (NMR) spectroscopy, the authors elucidated the structure of the 32-mer MC-aptamer to be a G-quartet (or quadruplex) containing a duplexed end, as illustrated schematically in FIG. 1A. DT-aptamer (SEQ ID NO:3), on the other hand, was identified by Tasset et al. ((1997) J. Mol. Biol. 272:688-698; ligand 60-18[27]) using free thrombin in solution as the target during the SELEX procedure. The authors proposed a similar quadruplex/duplex structure for the DT-aptamer based on the presence of the core sequence forming a G-quartet and base pairing in the duplex region.

The first aptamer binding to α-thrombin was described by Bock et al. ((1992) Nature 355:564-566) and was isolated using immobilized thrombin on a concanavalin A-agarose support using the SELEX method. This aptamer is a 15-mer containing just a quadruplex, without a duplexed end and bound to the fibrinogen-recognition exosite or the anion binding exosite (Wu et al. (1992) J. Biol. Chem. 267:24408-24412; Paborsky et al. (1993) J. Biol. Chem. 268:20808-20811) on thrombin. Macaya et al. ((1995) Biochemistry 34:4478-4492) reported competition between an aptamer that is similar to the MC-aptamer containing quadruplex/duplex structure, and the 15-mer aptamer containing just the quadruplex (Bock et al. (1992) Nature 355:564-566), suggesting that the aptamer similar to MC-aptamer also recognizes the fibrinogen-recognition exosite on thrombin. On the other hand, Tasset et al. ((1997) J. Mol. Biol. 272:688-698) reported poor competition between the DT-aptamer and the 15-mer aptamer, suggesting that the DT-aptamer is binding to a different site on thrombin. Furthermore, these authors presented experimental evidence to support that DT-aptamer binds to the heparin-recognition exosite on thrombin, since it competes well with an RNA aptamer (Kubik etal. (1994) Nucleic Acids Res. 22:2619-2626) known to bind to that site. These studies indicated that the two aptamers, DT-aptamer (SEQ ID NO:3) and MC-aptamer (SEQ ID NO:2), may have non-overlapping binding sites on thrombin, in spite of their very similar predicted secondary structure, a G-quartet connected to a short duplex region through an internal bulge. This observation provoked the design of a sandwich assay to detect thrombin using these two aptamers which is illustrated in FIG. 1B and described in Example 2.

With reference to Example 2, a single-step-binding assay format was employed that utilizes microsphere particle analysis in flow cytometry. Flow cytometry data analysis was carried out using the Luminex Flow Metrix™ analysis software program. This program allows multiplexed data acquisition and analysis for microsphere-based assays carried out on flow cytometry. (Fulton et al. (1997) Clin. Chem. 43:1749-1756). This platform utilizes microsphere particles dyed with different ratios of red- and orange-emitting fluorescent dyes. Each category of particles has a unique ratio of red- and orange-emitting fluorescent dyes enabling the software program to distinguish them using the ratio of red and orange fluorescence in FL3 and FL2 channels, respectively. This leaves the FL1 channel for data acquisition using green-emitting fluorophores, such as fluorescein. Microsphere particles (particles called Lumavidin™) containing a unique ratio of red and orange fluorescent dyes and streptavidin conjugated to the particle surface were used. The capture aptamer synthesized with biotin at the 3′ terminus was attached to Lumavidin™ particles using a streptavidin-biotin interaction as described in Example 1. To alleviate steric crowding between capture aptamers on the particle surface, a linker consisting of six thymidine residues was incorporated between the aptamer and the biotin residue at the 3′ terminus. Under saturation conditions, a loading of approximately 5 attomoles of a biotinylated aptamer per bead (data not shown) was achieved, a yield that is in agreement with previously reported value for similar beads with carboxyl groups, but derivatized using EDC chemistry. (Fulton et al. (1997) Clin. Chem. 43:1749-1756). The other aptamer that serves as the reporter in the sandwich assay was conjugated to fluorescein. The assay was performed by mixing the capture microsphere particles with the protein and the reporter aptamer for 15 minutes at 37° C. in the assay buffer, followed by measuring fluorescence on a fixed number of beads in a flow cytometer.

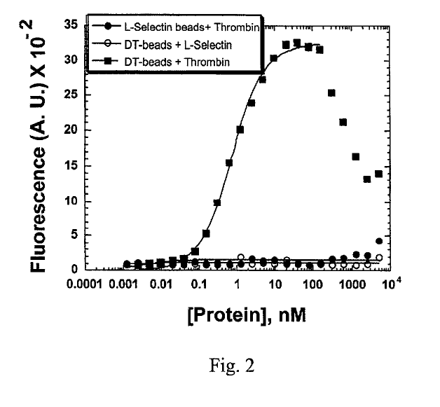

FIG. 2 shows the results of the one-step sandwich assay designed to detect thrombin. In this configuration, the DT-aptamer was immobilized on DT-beads and the fluoresceinated MC-aptamer in solution served as the reporter. The fluorescence signal increases with increasing thrombin concentration (FIG. 2; squares), indicating the formation of the sandwich. The signal increases up to a certain protein concentration in a dose-dependent manner, and then suddenly decreases. The assay detects thrombin between 0.1-<100 nM concentration, before the signal starts to decrease. The phenomenon of decreasing signal for the protein concentration that exceeds the binding capacity of the capture and the reporter ligands is referred to as the “hook effect,” and has been commonly observed in one-step immunoassays. (Fernando and Wilson (1992) J. Immunol. Methods 151:47-66; Nomura et al. (1983) J. Immunol. Methods 56:13-17; Utgaard et al. (1996) Clin. Chem. 42:1702-1708). As observed here, similar to antibody-based one-step immunoassays, the one-step aptamer-based assay also exhibits the “hook effect.” Control experiments included in FIG. 2 (circles) demonstrate the specificity of the assay. No signal above the background is observed when L-Selectin is used as the target for the DT-beads (FIG. 2; open circles), indicating that these two thrombin-specific aptamers are unable to form a sandwich with L-Selectin protein. Furthermore, when DT-beads are replaced with beads conjugated with an aptamer specific for L-Selectin (LS-beads), no fluorescence above the background is observed (FIG. 2; closed circles), indicating the lack of formation of a sandwich on LS-beads with fluoresceinated MC-aptamer.

The use of several different derivatives of the two aptamers at the reporter end of the assay were studied. The primary structures of different reporter constructs used in the study are set forth in Table 1 (SEQ ID NOS:4-13). With reference to Table 1, constructs indicated by the prefix MC are derived from the MC-aptamer (SEQ ID NO:2), whereas those indicated by the prefix DT are based on the DT-aptamer (SEQ ID NO:3). Each construct contains a single fluorescein group. The “X” in dimeric constructs shows the glycerol backbone, whose structure is illustrated at the bottom of table, which allowed for the symmetric synthesis of the monomeric arms (branching-phosphoramidite).

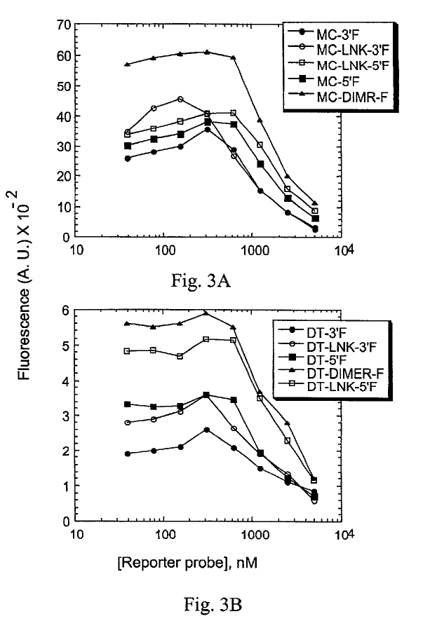

As noted above, each derivative studied contained a single fluorescein molecule. In monomeric aptamer derivatives, the fluorophore is attached either to the 5′ end or to the 3′ end with or without a six-thymidine linker. In dimeric forms, the fluorophore is present in the middle of the two monomeric units and flanked by thymidine linkers. Derivatives of each class of reporter aptamer (MC and DT) were studied using beads containing the opposite class of aptamer. For example, derivatives of MC-aptamer were studied using DT-aptamer on beads (DT-beads). The results of the analysis of these derivatives are summarized in FIGS. 3A and B. The following observations can be made from these results.

1. The signal intensities of the two possible configurations of the bead-based assay are different by about 10-fold; the use of the DT-aptamer as the capture and the MC-aptamer as the reporter gave a brighter signal than the signal observed with the opposite configuration of the assay. Table 2 illustrates the experimental ratio of fluorescein:DNA for each reporter construct whose structure is shown in Table 1. Although the theoretical value of this ratio is 1 for each construct, experimental values varied from 0.66-1.02. There is no direct correlation between the observed signal and the amount of fluorescein present in each reporter construct. DNA concentrations in Table 2 were based on 1 A₂₆₀ U=33 μg/mL in 50 mM sodium phosphate (pH 9.0) buffer. An extinction coefficient of 76,900 M⁻¹ cm⁻¹ at 491 nm was used for fluorescein tethered to DNA in 50 mM sodium phosphate (pH 9.0) buffer.

2. In both configurations reporter aptamers with fluorescein directly attached to the 5′ terminus gave a better signal than the ones with fluorescein at the 3′ terminus (FIG. 3, closed squares vs close circles).

3. The placement of the thymidine linker between fluorescein and the aptamer improved signal intensity. This is true whether the fluorophore is attached to the 3′ or to the 5′ terminus (compare, closed circles and squares with open circles and squares). The attachment of a linker between a fluorophore and an aptamer to improve the signal intensity in flow cytometry is consistent with the observation made in previous studies using an entirely different set of aptamers and targets. (Davis et al. (1996) Nucleic Acids Res. 24:702-706).

4. In all cases, the fluorescent signal decreased with the increase in concentration of the reporter aptamer, and optimal signal intensity was observed with relatively low concentrations of the reporter aptamer. The signal is expected to remain constant if the binding of the two aptamers, the capture and the reporter, is mutually exclusive. However, the observation that the signal decreases at very high concentrations suggests that there may be some degree of competition between the two types of aptamers at high concentrations, leading to the loss of thrombin on beads at high reporter aptamer concentrations. This result is analogous to the poor competition observed by Tasset etal ((1 997) J. Mol. Biol. 272:688-698) between DT-aptamer and the 15-mer anti-thrombin aptamer. Although nonspecific electrostatic interactions between thrombin and anionic oligonucleotides could be the possible cause for competition, the presence of tRNA at a very high concentration (100 μM) in these reactions is likely to rule out that possibility. Since the competitor constant, K_{c}, for interaction of nonspecific DNA with the heparin-recognition exosite of thrombin is 5.3 μM (Tasset et al. (1997) J. Mol. Biol. 272:688-698), 20-fold higher tRNA concentration is very likely to saturate nonspecific oligonucleotide interaction with thrombin. Alternatively, the decrease in signal at high concentration of the reporter probe could be independent of possible competitive binding of the two probes, but simply could be due to the transfer of captured thrombin from particles onto the reporter ligands that are in solution (discussed below). To achieve maximal signal intensity to increase the sensitivity of the assay, the reporter probe concentration should be kept low and fall within the plateau region observed in FIG. 3.

5. In both sets of reporter probes the dimer constructs gave the highest signal, possibly due to avidity in the dimer compared to monomers. Antibodies used in sandwich immunoassays are typically bivalent and hence have the advantage of avidity in target binding. Aptamers are generally monovalent and lack avidity contribution, yet they have been successful in various diagnostic assays (Jayasena (1999) Clin. Chem. 45:1628-1650), including sandwich assays as demonstrated here. Although aptamers are typically monovalent, dimeric (Davis et al. (1996) Nucleic Acids Res. 24:702-706; Ringquist and Parma (1998) Cytometry 33:394-405) or multimeric forms (Davis etal. (1998) Nucleic Acids Res. 26:3915-3924) of aptamers can be easily obtained. In both cases, the dimeric construct gave a better signal than monomeric forms, presumably due to enhanced resident time of the dimer on the target protein aided by avidity.

In order to determine the possible reason for the difference in the performance of the assay configured on the two types of beads, competition experiments were carried out to understand relative affinities of the two aptamers to thrombin as described in Example 3. The competition experiments were carried out under the identical conditions in which the assays were run. In the competition experiments, a fixed number of beads conjugated to the capture aptamer were incubated with a fixed concentration of thrombin, a fixed concentration of the fluoresceinated reporter aptamer and varying concentration of the unlabeled reporter aptamer in solution. These self-competition assays revealed apparent competitor constants (K_{c}s) that are equal to the apparent dissociation constants (K_{d}s). It was noticed that the affinity of the DT-aptamer was approximately 50-fold higher than that of the MC-aptamer under the conditions of the assay; K_{d} values: DT-aptamer=0.3 nM, MC-aptamer=14.6 nM (data not shown). These values are in agreement with those reported previously for these two types of aptamers. Tasset et al. ((1997) J. Mol. Biol. 272:688-698) had obtained a K_{d} of 0.5 nM for a DT-aptamer consisting of a shorter stem than the one used in this study. Macaya et al. ((1995) Biochemistry 34:4478-4492) have estimated K_{d} values of approximately 10-25 nM for a group of aptamers that included the MC-aptamer studied here. The poor signal observed in the assay configured on MC-beads (FIG. 3B) could be a direct result of the low affinity of the capture aptamer. In other words, the low affinity of the MC-aptamer may contribute to the poor efficiency in capturing thrombin on beads.

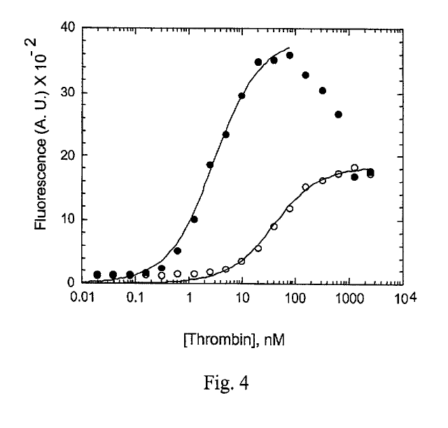

Upon determining that the aptamer-based sandwich assay can be used to detect thrombin in assay buffer, the performance of the assay in detecting thrombin in a complex background that contains a host of proteins and other biomolecules at various concentrations was then investigated. Human plasma and human urine were chosen for this experiment, as described in Example 2. The assay was initiated by adding a mixture of DT-beads and MC-5′-LNK-F to either plasma or urine to which thrombin was spiked at varying concentrations. Detection of thrombin spiked into urine using the method described herein is shown in FIG. 4 (closed circles). This result is very similar to that obtained with thrombin in the assay buffer, demonstrating that the assay specifically detects thrombin in a complex background that contains other biomolecules. The detection of thrombin spiked into human plasma, however, is quite different from that observed with thrombin spiked into either assay buffer or human urine (FIG. 4; open circles). The response curve in plasma is shifted to the right, indicating that only high concentrations of thrombin could be detected in plasma. This result is likely due to the propensity of thrombin to associate with different molecules in plasma. It is known that thrombin does not naturally exist in its free form, but is derived from the cleavage of prothrombin on demand. Overall, the results shown in FIG. 4 demonstrate that the aptamer-based sandwich assay can be extended to measure the target analyte in a complex background.

To study the feasibility of multiplexed analysis, at least two different assays aimed at detecting two different targets are required. For this purpose, an aptamer-based assay was designed that detects human L-Selectin-Ig chimera. This assay is based on a high-affinity DNA aptamer specific for human L-Selectin. The identification and the characteristics of the L-Selectin aptamer have been previously reported. It binds human L-Selectin with a K_{d} of 1.8 nM and has the potential to fold into a stem-loop structure with an internal bulge. (Hicke etal. (1996) J. Clin. Invest. 98:2688-2692). Similar to the DT-aptamer, the L-Selectin aptamer synthesized with a biotin molecule at the 3′ terminus was immobilized on Lumavidin™ particles to obtain LS-beads as described in Example 1. The red/orange fluorescent dye ratio of the particles used to immobilize L-Selectin aptamer is different from that of the particles used to conjugate thrombin-specific DT-aptamer. The use of two distinct categories of microsphere particles allows simultaneous analysis of them in flow cytometry using the FlowMetrix™ software program.

Due to the absence of a second aptamer that recognizes L-Selectin at a non-overlapping site, L-Selectin-Ig chimera was used as the target protein which could be detected with Protein A conjugated to a fluorescent reporter. Protein A is a 42-kDa protein isolated from the cell walls of Staphylococcus aureus and is known to bind to the Fc region of an antibody. (Langone (1982) J. Immunol. Methods 51: 3-15). The sandwich assay designed for detecting L-Selectin involved capturing L-Selectin-Ig chimera by the DNA aptamer immobilized on microsphere particles (LS-beads) and detecting the captured protein with Protein A-Alexa conjugate.

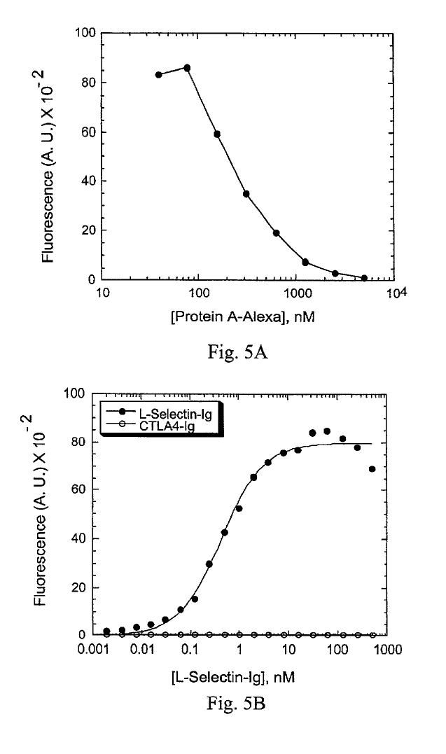

The signal intensity of the assay that employed an aptamer and Protein A as a function of the concentration of the reporter ligand is depicted in FIG. 5. FIG. 5A shows the results obtained with varying concentrations of Protein A-Alexa conjugate on a fixed amount of LS-beads incubated with a constant concentration of L-Selectin-Ig protein chimera. Similar to the observation made for thrombin assay, the signal in the L-Selectin assay also decreases with the increase in the reporter ligand concentration. Since the aptamer that binds to L-Selectin had been isolated using L-Selectin-Ig chimera presented on Protein A-agarose beads, the probability that the aptamer and Protein A share overlapping binding sites is extremely low. Based on this view, it is unlikely that the observed decrease in signal with increasing concentration of Protein A-Alexa could be due to the competition between the two ligands, but likely is due to the transfer of L-Selectin-Ig captured by aptamers on microspheres to Protein A-Alexa in solution. Based on the titration curve in FIG. 5A, 80 nM Protein A-Alexa concentration were chosen for the sandwich assay. FIG. 5B (closed circles) shows the results of the one-step sandwich assay for detection of the L-Selectin-Ig chimera. Similar to the thrombin assay, the detection range of the L-Selectin assay is 0.1-100 nM, and also suffers from the high-dose “hook effect” phenomenon. The specificity of the L-Selectin assay was tested by replacing L-Selectin-Ig chimera with CTLA4-Ig chimera, a fusion protein that is recognized by the reporter ligand, Protein A-Alexa conjugate, but not by the capture aptamer on microsphere particles. As shown in FIG. 5B (open circles), there is no signal above the background when CTLA4-Ig chimera is used in place of L-Selectin-Ig chimera, indicating that the aptamer, specific for L-Selectin, does not capture CTLA4-Ig chimera on microsphere particles.

FlowMetrix™ software program that analyzes the signal from a flow cytometer allows for multiplexed analysis of particle-based assays. Multiplexing is based on simultaneous analysis of beads that are dyed in different ratios of red and orange fluorophores. While red and orange fluorescence are used for bead classification, the green fluorescence is used for measuring analytes on beads. The two assays studied were configured on two types of beads with distinct red/orange fluorescent dye ratio and utilize reporter ligands conjugated to a green fluorophore. Hence, they satisfy the basic requirements for multiplexing using FlowMetrix™ software.

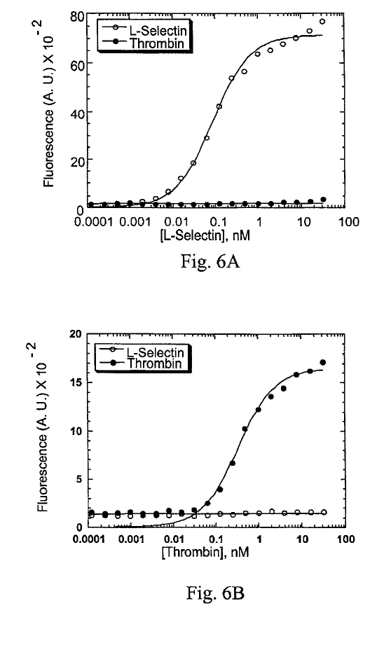

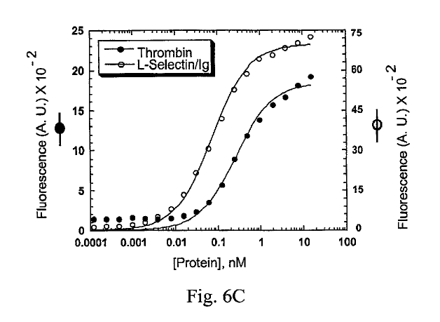

When designing assays for multiplexed analysis, several factors must be taken into account and optimized such that each assay will perform effectively when all the components are present in the same solution. One factor to be considered is the buffer, which must be compatible with both assays that are to be performed. In the present study, the individual sandwich assays were carried out in two different buffers. The thrombin assay was carried out in TBSM-BTT buffer, whereas the L-Selectin buffer was carried out in SHMCK-BTT buffer. These buffers were chosen on the basis of buffers used for the aptamer identification during the SELEX process. In preliminary studies it was determined that the L-Selectin assay did not work well in the TBSM-BTT buffer, presumably due to the lack of calcium ions, which are required for the recognition of L-Selectin by the L-Selectin aptamer. (O'Connell et al. (1996) Proc. Natl Acad. Sci. USA 93: 5883-5887). A common buffer (TBSMC), that permitted both assays to perform well, was therefore, identified and used for multiplexed analyses. Multiplex assays were initiated with a mixture of two microspheres coupled to either the DT-aptamer or the L-Selectin aptamer and a mixture of fluoresceinated MC-aptamer and Protein A-Alexa. Varying concentrations of: a) thrombin alone (FIG. 6A); b) L-Selectin-Ig chimera alone (FIG. 6B); and c) both proteins (FIG. 6C) were added to the above mixture, incubated for 15 minutes at 37° C. and fluorescence on each type of bead was measured simultaneously. As indicated in FIG. 6, performance of each assay is independent of the other. There is virtually no interference from LS-beads and Protein A-Alexa (FIG. 6A) towards the detection of thrombin. Likewise, no interference from the presence of DT-beads and the fluoresceinated MC-aptamer is observed on the detection of L-Selectin (FIG. 6B). The difference in the signal intensity between the two assays can be attributed to the intrinsic fluorescence characteristics of the fluorophores used; Alexa is a brighter fluorophore than fluorescein. The independent nature of the two assays makes it possible to simultaneously detect both proteins in a single tube as shown in FIG. 6C, demonstrating multiplexed analysis of proteins using aptamers.

Multiplexed analysis can also be performed on spatially addressable test sites. DNA chip arrays in which known DNA sequences are immobilized on a solid surface to capture and detect unknown sequences have been designed for genomic applications. Analogous to DNA arrays, aptamer arrays printed on an appropriate surface can be designed to analyze a large number of protein targets to analyze the amounts and types of proteins present in a cell, a tissue or a biological fluid. Protein analysis or proteomics is expected to provide the phenotype or the true picture of genes that are expressed at the functional level in a cell, rather than the genotype or the type of genes that have the potential to be expressed in a cell. Since phenotypic information is more meaningful for diagnostic purposes, tools that are at disposal for proteomics applications would become more and more valuable. Arrays based on antibodies are also being developed for analyzing a large number of proteins. (Dove (1999) Nature Biotechnol. 17:233-236), but aptamer arrays are expected to be attractive for this application for the reasons outlined. (Jayasena (1999) Clin. Chem. 45:1628-1650).

Geometrically addressable arrays of nucleic acid sequences are becoming more and more popular in the analysis of nucleic acids for high-throughput genomic applications. Analogous to DNA arrays for genomic applications, one could envision developing arrays of antibodies for analyzing proteins for proteomics. Aptamers that specifically bind to their target proteins with high-affinity provide another class of reagent that could be used for analyzing proteins on an array format. As a prelude to this effort, the capture and detection of thrombin by the two aptamers on a membrane was investigated as described in Example 5.

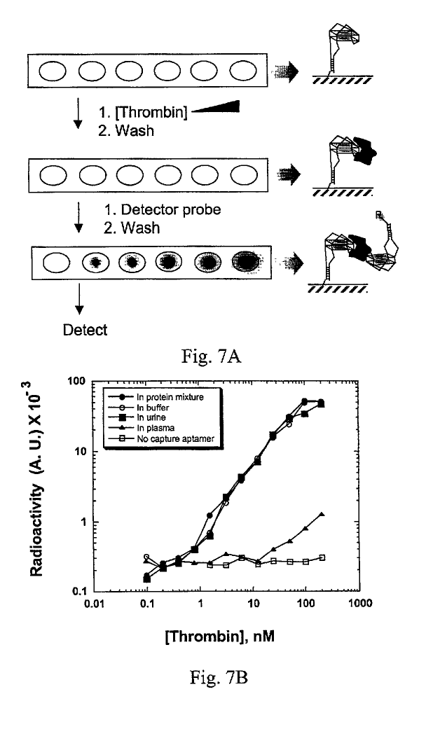

A Biodyne C membrane containing carboxylic groups on the surface was chosen as the solid matrix to attach the DT-aptamer for capturing thrombin. For this purpose the DT-aptamer was synthesized with a primary amine group at the 3′ end with a six-thymidine linker and reacted with the carboxylic groups on the membrane that have been activated with EDC. This procedure led to the attachment of the aptamer at a density of between 5-10 pmoles/mm² (data not shown). The membrane derivatized with the DT-aptamer was then exposed to increasing concentrations of thrombin spiked into the following solutions: a) assay buffer alone, b) assay buffer containing a mixture of known proteins, c) human plasma and d) human urine. After washing the membrane to remove unbound protein(s), thrombin captured by the aptamer was detected with radiolabeled MC-DIMER aptamer (FIG. 7). With reference to FIG. 7B, very similar results were obtained when thrombin was in the assay buffer (open circles), assay buffer containing a protein mixture (closed circles), and human urine (closed squares), indicating that the presence of nonspecific proteins in the test sample did not interfere with thrombin detection. The detection range of the assay is 0.1-100 nM of thrombin. The result obtained with the samples containing thrombin-spiked plasma (triangles) is different from the above three cases. Similar to the result observed in the bead format (FIG. 5; closed circles), the response curve in plasma is shifted to the right. This is presumably due to the apparent unavailability of thrombin for the capture and detection in plasma owing to the propensity of thrombin to associate with plasma proteins. The control experiment carried out in the absence of the DT-aptamer immobilized on the membrane gave no signal above the background (FIG. 7; open squares). This result indicates that the observed signal is a result of thrombin captured by the aptamer and not due to the possible nonspecific adsorption of the protein on the membrane.

Proteins that exist in a cell extract or a biological fluid have to be separated for homogeneity at each location, preferably by two-dimensional gel electrophoresis, transferred to a membrane and then detected with appropriate library of aptamers. This rather cumbersome approach can be simplified by capturing the individual protein with its cognate aptamer immobilized on a solid surface and detecting it with an appropriate reagent that reacts exclusively with proteins, but not with nucleic acids. This study illustrates the capture of a specific protein in a mixture of proteins by the cognate aptamer immobilized on a membrane and the detection of the captured protein by a second aptamer. Replication of this process for a set of proteins, rather than one as demonstrated here, using their cognate aptamers immobilized on a surface and detecting them using appropriate secondary ligands will be the aptamer array for proteomics. Development of a universal reagent that exclusively tags proteins could replace the secondary ligand specific for each protein, and further simplify the approach. Alternatively, a physical method such as evanescent wave-induced fluorescence anisotropy could also be used without a secondary ligand, but with fluorescent-labeled aptamer immobilized on a solid surface. (Potyrailo et al. (1998) Anal. Chem. 70:3419-3425).

The following examples are provided to explain and illustrate the present invention and are not to be taken as limiting of the invention.

### EXAMPLES

Materials. DNA aptamers synthesized by standard solid phase oligonucleotide synthesis and purified by reverse phase high pressure liquid chromatography (HPLC) were purchased from Operon Technologies, Inc. (Alameda, Calif.). Human α-thrombin was purchased from Enzyme Research Laboratories (South Bend, Ind.). L-Selectin protein was purchased from R & D Systems (Minneapolis, Minn.). L-Selectin-Ig and CTLA4-Ig fusion proteins were generous gifts from David Parma and Alan Korman, respectively at NeXstar Pharmaceuticals, Inc. Protein A-Alexa conjugate was purchased from Molecular Probes (Eugene, OR). LumAvidin™ microsphere particles were purchased from Luminex Corporation (Austin, Tex.). Biodyne C nylon membrane (0.45 μm diameter) was bought from Pall Gelman Laboratories (Ann Arbor, Mich.). EDC (1-ethyl-3-(dimethylaminopropyl)carbodiimide hydrochloride) was purchased from Pierce Chemicals (Rockford, Ill.). All other reagents used were of analytical grade.

### Example 1

### Preparations of Capture Microspheres

Aptamers that serve as the capture in sandwich assays were synthesized with biotin at the 3′ end and a six-thymidine linker between biotin and the aptamer sequence. Lumavidin™ microspheres 8087 and 8047 were used to attach a thrombin-specific aptamer (either DT-Aptamer or MC-aptamer) and the L-Selectin-specific aptamer, respectively. The L-Selectin aptamer, 5′-TAGCCAAGGTAACCAGTACAAGGTGCTAMCGTAATGGCTTCGGCTTAC-3′ (SEQ ID NO:1), used in this study was described previously (O'Connell et al. (1996) Proc. Natl Acad. Sci. USA 93: 5883-5887). Conjugation of biotinylated aptamers was carried out according to manufacturer's instructions with minor modifications. Briefly, a stock suspension of microsphere particles (1×10⁷ beads/mL) was sonicated for one minute at ambient temperature in a bath sonicator to disrupt aggregates. An aliquot of particles (0.2×10⁶) were washed two times in a buffer consisting of 100 mM Tris-HCl (pH 8.0) and 0.01% (w/v) SDS. Particles were then resuspended in the same buffer in 100 μL volume and mixed with 300 picomoles of a biotinylated oligonucleotide. The mixture was incubated at ambient temperature for 45 minutes with gentle agitation. Particles conjugated with the oligonucleotide were rinsed five times in the above buffer containing 0.1% (v/v) Tween-20 instead of SDS to remove unconjugated oligonucleotides, and finally resuspended in the same buffer at a concentration of 1×10⁶ particles/mL. DT-beads, MC-beads and LS-beads represent microsphere particles conjugated with DT-Aptamer, MC-Aptamer and L-Selectin aptamer, respectively.

### Example 2

### One-Step Sandwich Assays

Varying concentrations of human α-thrombin were prepared in the thrombin assay buffer consisting of 50 mM Tris-HCl (pH 7.5), 100 mM NaCl, 2 mM MgCl₂, 0.2% (w/v) BSA, 100 μM tRNA and 0.1% (v/v) Tween-20 (TBSM-BTT buffer). A 10 μL aliquot of thrombin in TBSM-BTT buffer was mixed with 10 μL of 600 nM reporter aptamer solution and 10 μL of bead suspension (2×10³ beads) in the same buffer. The three components were mixed briefly by gentle vortexing, incubated at 37° C. for 10 minutes, mixed with 200 μL of the assay buffer and analyzed by FacsCaliber flow cytometer (Becton Dickinson) using FlowMetrix™ software program (Luminex). Fluorescence on 300 beads was collected for each data point carried out in duplicates. The average value of fluorescence intensity was used for data analysis.

Detection of thrombin either in human plasma or in human urine was carried out in the same manner except that dilution of thrombin was carried out in either freshly prepared plasma or in fresh urine. Equal volume of thrombin in either human plasma or in human urine was mixed with an equal volume of bead suspension mixed with the reporter aptamer in 2× TBSM-BTT buffer.

L-Selectin assays were carried out in a manner similar to that described for the thrombin assay, but in a buffer consisting of 20 mM HEPES (pH 7.4), 150 mM NaCl, 1 mM CaCl₂, 1 mM MgCl₂, 0.2% (w/v) BSA, 100 μM tRNA and 0.1% Tween-20 (SHMCK-BTT buffer). A 30 μL reaction contained 2×10³ LS beads, 80 nM protein A-Alexa and a given concentration of L-Selectin-Ig chimera. These reactions were incubated for 10 minutes at 37° C., and diluted with 200 μL of SHMCK-BTT buffer immediately prior to flow cytometry analysis.

### Example 3

### Self-Competition Assays

Self-competition assays were carried out by incubating 2×10³ beads (MC-beads) in TBSM-BTT buffer containing 30 nM thrombin, 200 nM reporter aptamer (DT-5′-LNK-F) and increasing concentrations of the competitor (unlabeled DT-Aptamer) for 10 minutes at 37° C. After adding 200 μL of TBSM-BTT buffer, fluorescence on 300 beads was measured. To analyze self-competition of MC-Aptamer, the opposite configuration of the assay employing DT-beads and unlabeled MC-Aptamer and MC-5′-LNK-F was used.

### Example 4

### Multiplexed Analysis

Multiplexed analysis of proteins was carried out using a mixture of two types of capture beads. The assay employed LS-beads and DT-beads prepared on Lumavidin™ microspheres 8047 and 8087, respectively, and was carried out in multiplex buffer that contained TBSM-BTT supplemented with 1 mM CaCl₂ (TBSMC-BTT buffer). A mixture of LS-beads and DT-beads each containing 2×10³ beads was incubated in 30 μL of TBSMC-BTT buffer containing 200 nM MC-5′-LNK-F, 80 nM protein A-Alexa, and varying concentrations of one of the following: 1) L-Selectin-Ig chimera; 2) thrombin; or 3) a mixture of L-Selectin-Ig chimera and thrombin. After incubating at 37° C. for 10 minutes, a 200 μL aliquot of TBSMC-DTT buffer was added and fluorescence was measured on 500 events on the two types of beads.

### Example 5

### Two-Step Sandwich Assay on a Nylon Membrane

All reactions on Biodyne C membrane were carried out using a 96-well filter manifold (Bio-Rad, Calif.). DT-aptamer synthesized with a primary amine group at the 3′ terminus was coupled to carboxylic groups on the nylon membrane by EDC activation. (Zhang et al. (1991) Nucleic Acids Res. 19:3929-3933). Carboxylic groups on the surface of Biodyne C membrane were activated by reacting with EDC in a solution containing 200 mM imidazole, 20 mM MES (pH 4.5) and 20% (w/v) EDC for 20 minutes at room temperature. The EDC solution was removed under suction and the activated membrane was wetted with 50 μL of distilled water before adding 25 picomoles of the amine-modified DT-aptamer resuspended in 0.5 M sodium borate buffer (pH 8.5) to each well. The coupling reaction was carried out for 1 hour at room temperature. The membrane was washed three times, each with 200 μL volume of a buffer consisting of 50 mM Tris-HCl (pH 7.5) and 100 mM NaCl to remove unreacted oligonucleotides. Carboxylic groups that remained activated with EDC, but did not react with oligonucleotide, were neutralized by incubating the membrane in 200 μL of 0.1 N NaOH solution for 10 minutes. The membrane was then washed four times with a buffer consisting of 50 mM Tris-HCl (pH 7.5), 100 mM NaCl and 2 mM MgCl₂ and finally with 50 mM Tris-HCl (pH 7.5), 100 mM NaCl and 2 mM MgCl₂, 0.2% (w/v) BSA, 0.02% (w/v) Tween-20 and 100 μM Heparin (TBSM-BHT buffer).

The assay was initiated by wetting the membrane with TBSM-BHT buffer. Varying concentrations of thrombin prepared in TBSM-BHT buffer were added to each well and incubated for 20 minutes at room temperature. The membrane was washed four times with TBSM-BHT buffer to remove thrombin that was not captured by the aptamer. Either radiolabeled or fluoresceinated detector aptamer (MC-DIMR-F) was added to each well and incubated for 20 minutes at room temperature. Excess detector aptamer was removed from the membrane by washing four times with TBSM-BHT buffer. The signal on the membrane was quantified with either Phosphorimager (for radiolabeled detector aptamer) or Fluorimager (for fluoresceinated detector aptamer). For detecting thrombin in complex background, such as plasma or urine, thrombin spiked into 2× TBSM-BHT buffer was mixed with equal volume of freshly prepared human plasma or fresh human urine and used in the assay as described above. The average value of radioactivity or fluorescence obtained from duplicate reactions was plotted against the protein concentration.

TABLE 1

| Name | Aptamer sequence | SEQ ID NO: |
|---|---|---|
| MC-3′-F | 5′-GTAGTCACTGGTTGGTGAGGTTGGGTGACTAC-F-3′ | 4 |
| MC-3′-LNK-F | 5′-GTAGTCACTGGTTGGTGAGGTTGGGTGACTAC-TTTTTT-F-3′ | 5 |
| MC-5′-F | 5′-F-GTAGTCACTGGTTGGTGAGGTTGGGTGACTAC-3′ | 6 |
| MC-5′-LNK-F | 5′-F-TTTTTT-GTAGTCACTGGTTGGTGAGGTTGGGTGACTAC-3′ | 7 |
| MC-DIMR-F | 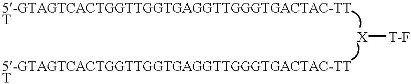 | 8 |
| DT-3′-F | 5′-GCTTAGTCCGTGGTAGGGCAGGTTGGGGTGACTAAGC-F-3′ | 9 |
| DT-3′-LNK-F | 5′-GCTTAGTCCGTGGTAGGGCAGGTTGGGGTGACTAAGC-TTTTTT-F-3′ | 10 |
| DT-5′-F | 5′-F-GCTTAGTCCGTGGTAGGGCAGGTTGGGGTGACTAAGC-3′ | 11 |
| DT-5′-LNK-F | 5′-F-TTTTT-GCTTAGTCCGTGGTAGGGCAGGTTGGGGTGACTAAGC-3′ | 12 |
| DT-DIMR-F | 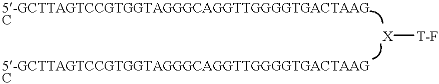 | 13 |
| | 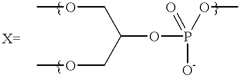 | |

TABLE 2

| | | | [Fluorescein] |
|---|---|---|---|
| Aptamer | [DNA] μM | [Fluorescein] μM | [DNA] |
| MC-3′-F | 190.9 | 161.4 | 0.85 |
| MC-3′-LNK-F | 198.7 | 195.0 | 0.98 |
| MC-5′-F | 202.5 | 185.3 | 0.90 |
| MC-5′-LNK-F | 190.4 | 142.5 | 0.75 |
| MC-DIMR-F | 151.5 | 101.2 | 0.66 |
| DT-3′-F | 196.4 | 156.8 | 0.80 |
| DT-3′-LNK-F | 194.1 | 198.2 | 1.02 |
| DT-5′-F | 198.1 | 199.5 | 1.00 |
| DT-5′-LNK-F | 195.8 | 176.6 | 0.90 |
| DT-DIMR-F | 179.5 | 119.8 | 0.66 |

1. A method for detecting the presence of a target compound in a substance which may contain said target compound comprising:
a) exposing a substance which may contain said target compound to a capture molecule capable of binding to said target molecule, wherein said capture molecule is immobilized on a solid support;
b) removing the remainder of said substance from said capture molecule:target molecule complex;
c) adding to said capture molecule:target molecule complex a reporter molecule capable of binding to said target molecule; and
d) detecting said capture molecule:target molecule:reporter molecule complex;
wherein said capture molecule, said reporter molecule or both are a nucleic acid ligand to said target molecule.

2. The method of claim 1 wherein said reporter molecule comprises a detection system.

3. The method of claim 2 wherein said detection system is a nucleic acid ligand labeled with a fluorophore.

4. The method of claim 3 wherein said fluorophore is selected from fluorescein or Alexa.

5. The method of claim 1 wherein said solid support is selected from a microsphere particle or a membrane.

6. The method of claim 1 wherein said target molecule is a protein.

7. The method of claim 6 wherein said protein is selected from thrombin or L-Selectin.

8. The method of claim 1 wherein said capture molecule and reporter molecule are nucleic acid ligands.

9. The method of claim 1 wherein said capture molecule is a nucleic acid ligand and said reporter molecule is a protein.

10. The method of claim 1 wherein said capture molecule and reporter molecules bind to separate non-overlapping sites on said target molecule.

11. The method of claim 1 wherein said reporter molecule binds to a site on said capture molecule:target complex.

12. The method of claim 1 wherein said substance is a biological fluid.

13. The method of claim 12 wherein said biological fluid is selected from plasma or urine.

14. The method of claim 1 wherein said detection is achieved by flow cytometry.

15. A method for detecting the presence of a target compound in a substance which may contain said target compound comprising:
a) identifying a nucleic acid ligand from a candidate mixture of nucleic acids, said nucleic acid ligand being a ligand of said target compound, by the method comprising:
i) contacting the candidate mixture with said target compound, wherein nucleic acids having an increased affinity to said target relative to the candidate mixture may be partitioned from the remainder of the candidate mixture;
ii) partitioning the increased affinity nucleic acids from the remainder of the candidate mixture;
iii) amplifying the increased affinity nucleic acids to yield a ligand-enriched mixture of nucleic acids; and
iv) identifying said nucleic acid ligand;
b) exposing a substance which may contain said target compound to a capture molecule capable of binding to said target molecule, wherein said capture molecule is immobilized on a solid support;
c) removing the remainder of said substance from said capture molecule:target molecule complex;
d) adding to said capture molecule:target molecule complex a reporter molecule capable of binding to said target molecule; and
e) detecting said capture molecule:target molecule:reporter molecule complex;
wherein said capture molecule, said reporter molecule are a nucleic acid ligand to said target molecule identified by the method of step (a).

16. The method of claim 15 wherein said reporter molecule comprises a detection system.

17. The method of claim 16 wherein said detection system is a nucleic acid ligand labeled with a fluorophore.

18. The method of claim 17 wherein said fluorophore is selected from fluorescein or Alexa.

19. The method of claim 15 wherein said solid support is selected from a microsphere particle or a membrane.

20. The method of claim 15 wherein said target molecule is a protein.

21. The method of claim 20 wherein said protein is selected from thrombin or L-Selectin.

22. The method of claim 15 wherein said capture molecule and reporter molecule are nucleic acid ligands.

23. The method of claim 15 wherein said capture molecule is a nucleic acid ligand and said reporter molecule is a protein.

24. The method of claim 15 wherein said capture molecule and reporter molecules bind to separate non-overlapping sites on said target molecule.

25. The method of claim 15 wherein said reporter molecule binds to a site on said capture molecule:target complex.

26. The method of claim 15 wherein said substance is a biological fluid.

27. The method of claim 26 wherein said biological fluid is selected from plasma or urine.

28. The method of claim 15 wherein said detection is achieved by flow cytometry.

29. A method for detecting the presence of two or more target compounds in a substance which may contain said target compounds comprising:
a) exposing a substance which may contain said target compounds to capture molecules, wherein each capture molecule is capable of binding specifically to a corresponding target compound, wherein said capture molecules are immobilized on a solid support;
b) removing the remainder of said substance from said capture molecule:target molecule complexes;
c) adding to said capture molecule:target molecule complexes reporter molecules; wherein each reporter molecule is capable of binding specifically to a corresponding target molecule; and
d) detecting said capture molecule:target molecule:reporter molecule complexes;
wherein said capture molecules, said reporter molecules or both are a nucleic acid ligand to said target molecules.
